SEQUENCE LISTING (33 sequences)

SEQ ID NO: 1 - protein, 933 aa, Anas platyrhynchos
MTADEKRSLQCYRRYIERSLNPVYVLGNMTDWLPDELRERIRKEEERGVSGAAALFLDAVLQLEARGWFRGMLDAMLAAGYTGLAEAIENWDFSKLEKLELHRQLLKRIEATMLEVDPVALIPYISTCLIDRECEEIQQISENRSKAAGITKLIECLCRSDKEHWPKSLQLALDTTGYYRASELWDIREDNAKDVDSEMTDASEDCLEASMTYSEEAEPDDNLSENLGSAAEGIGKPPPVYETKKARSYQIELAQPAINGKNALICAPTGSGKTFVSILICEHHFQNMPAGRKAKVVFLATKVPVYEQQKNVFKHHFERQGYSVQGISGENFSNVSVEKVIEDSDIIVVTPQILVNSFEDGTLTSLSIFTLMIFDECHNTTGNHPYNVLMTRYLEQKFNSASQLPQILGLTASVGVGNAKNIEETIEHICSLCSYLDIQAISTVRENIQELQRFMNKPEIDVRLVKRRIHNPFAAIISNLMSETEALMRTIYSVDTLSQNSKKDFGTQNYEHWIVVTQRKCRLLQLEDKEEESRICRALFICTEHLRKYNDALIISEDARIIDALSYLTEFFTNVKNGPYTELEQHLTAKFQEKEPELIALSKDETNENPKLEELVCILDDAYRYNPQTRTLLFAKTRALVSALKKCMEENPILNYIKPGVLMGRGRRDQTTGMTLPSQKGVLDAFKTSKDNRLLIATSVADEGIDIVQCNLVVLYEYSGNVTKMIQVRGRGRAAGSKCILVTSKTEVVENEKCNRYKEEMMNKAVEKIQKWDEETFAKKIHNLQMKERVLRDSRRKEIKPKVVEGQKNLLCGKCKAYACSTDDIRIIKDSHHIVLGEAFKERYTTKPHKKPMQFDGFEKKSKMYCRNNNCQHDWGITVKYLTFDNLPVIKIKSFVMESTATGTQMDFQKWKSINSSLKNFDVEEMSNLYPPF

SEQ ID NO: 2 - protein, 927 aa, Taeniopygia guttata
MTAEEKQNLRFYRRYIERSLNPVYILSNMTEWLSDEMEERVRKEEEKGVTAAAALFLDAVLLLEAEGWFRGFQDALAAAGYTGLAEAIENWDFGKLEKLELHRQLLKRIESTMLEIDPVAIMPYINTCLIERECDEILQISEYRSKAAGITKLIECLCRSDKENWPKSLQLALDSAGYYNASELWNIREDNGKDVDGEMTDASENNFETIMTFSEEAECDNLSKNLSSVSECICESSSVYEPKKARSYQIELAQPAIDGKNTLICAPTGSGKTFVAVLICEHHFQNIPSGRKAKVVFLATKMPVYEQQKNVFRQHFERSGYFVQGICGETVANISVENVIQDSDIIVLTPQILVNIMDKGILSSLSIFTLMIFDECHNTAGNHPYNVLMTRYLDQKFDSSAKQLPQIVGLTASVGVGNAKSTKETVEHICTLCSYLDIQAISTVRENKQDLQRFANKPEIHIRWVKMRAQNRFADIISGLMSETEVLMRKIYSVDTISQINKIYFGTQRYEHWIVSTQKKCRLLQLEDKEKESSICRDLFICTEHLRKFNDALIISEDARIEDALAYLNEFFTNVKNGPYTELEKQLTEKFQEIEQELTALSKDESNENPKLEELACILDEAYHYNPQTRTILFAKTRALVAALKKWVEANPLLSHIKPDVMMGKGRRDRTVGMTLPMQKGVLDAFRNDKDIRLLIATSVADEGIDITECNLVVLYEYFGNVTKMIQVRGRGRAKDSKCILVTSKTEVVENEKLNTYKEEMMNAAIEKLQNWDETTFARKIRGLQMKEKMLRDSRKNETKHELVEGKKKLYCGKCKAYACSTDDIRIIKGSHHIVLGNAFQERYTTKPHRKPVQFDDFVKKSKMHCRNTECQHDWGIIVKYKIFDNLPVIKIRSFVLEDVESGSQMDFQKWRSINLSLKNFDEETCS

SEQ ID NO: 3 - protein, 925 aa, Homo sapiens [sequence omitted]

SEQ ID NO: 4 - DNA, 26 bp, Artificial Sequence
  Primer
gatcccagcaatgagaatcctaaact

SEQ ID NO: 5 - DNA, 22 bp, Artificial Sequence
  Primer
caatgtcaatgccttcatcagc

SEQ ID NO: 6 - DNA, 19 bp, Artificial Sequence
  Primer
cggccggcagagcccagcc

SEQ ID NO: 7 - DNA, 25 bp, Artificial Sequence
  Primer
gtgtaggagagtaatagatgcacta

SEQ ID NO: 8 - RNA, 21 nt, Artificial Sequence
  Oligonucleotide
ggggcugacccugaaguuccc

SEQ ID NO: 9 - DNA, 20 bp, Artificial Sequence
  Oligonucleotide
taatacgactcactataggg

SEQ ID NO: 10 - DNA, 38 bp, Artificial Sequence
  Oligonucleotide
gggaacttcagggtcagcccctatagtgagtcgtatta

SEQ ID NO: 11 - DNA, 24 bp, Artificial Sequence
  Primer
acaggtatgaccctcccaagccag

SEQ ID NO: 12 - DNA, 27 bp, Artificial Sequence
  Primer
catcccatttctggatcttttcaacag

SEQ ID NO: 13 - DNA, 24 bp, Artificial Sequence
  Primer
gtatgaccctcccaagccagaagg

SEQ ID NO: 14 - DNA, 26 bp, Artificial Sequence
  Primer
ctctgacttggatcattttggtcacg

SEQ ID NO: 15 - DNA, 28 bp, Artificial Seqeunce
  Primer
gagcaaagggaagtcattgataaattcc

SEQ ID NO: 16 - DNA, 24 bp, Artificial Sequence
  Primer
ggcctgcaacatagcaatttcatt

SEQ ID NO: 17 - DNA, 30 bp, Artificial Sequence
  Primer
gtgtatggaggaaaaccctatttcttaact

SEQ ID NO: 18 - DNA, 24 bp, Artificial Sequence
  Primer
ggagggtcatacctgttgtttgat

SEQ ID NO: 19 - DNA, 16 bp, Artificial Sequence
  Primer
ttccgcgccccatcaa

SEQ ID NO: 20 - DNA, 19 bp, Artificial Sequence
  Primer
gcctcttgcaccaccaact

SEQ ID NO: 21 - DNA, 23 bp, Artificial Sequence
  Primer
ggcatggacagtggtcataagac

SEQ ID NO: 22 - DNA, 16 bp, Artificial Sequence
  Probe
cacaatgccaaagttg

SEQ ID NO: 23 - DNA, 22 bp, Artificial Sequence
  Primer
gaccratcctgtcacctctgac

SEQ ID NO: 24 - DNA, 24 bp, Artificial Sequence
  Primer
agggcattytggacaaakcgtcta

SEQ ID NO: 25 - DNA, 24 bp, Artificial Sequence
  Primer
tgcagtcctcgctcactgggcacg

SEQ ID NO: 26 - DNA, 20 bp, Artificial Sequence
  Primer
gtttcggacatggggagtaa

SEQ ID NO: 27 - DNA, 23 bp, Artificial Sequence
  Primer
gcatacgatttcttcaactttgg

SEQ ID NO: 28 - DNA, 21 bp, Artificial Sequence
  Primer
tgcttgactggaaaggctact

SEQ ID NO: 29 - DNA, 24 bp, Artificial Sequene
  Primer
tcagtcaagaataaaccatgtgtg

SEQ ID NO: 30 - DNA, 3715 bp, Anas platyrhynchos
cggccggcagagcccagccatgacggcggacgagaagcggagcctgcagtgctaccgccgctacatcgagcggagcctcaacccggtctacgtgctgggcaacatgacggactggctgcccgacgagctgcgggagaggatccgcaaggaggaggagaggggggtgagcggcgccgccgcgctcttcctggacgccgtgctgcagctggaggcccgggggtggttccgagggatgctggacgcgatgctggccgcaggttacacaggactggcagaagcaattgagaactgggacttcagcaaactggaaaaactggagttacacagacagctgttgaagcggatagaggcaacaatgttagaagtcgacccagtagcgctcattccctatataagcacatgcctgatagacagggagtgtgaagagattcagcagattagtgaaaacagaagcaaagcagcaggcataactaaactcattgaatgtctctgtcggtcggataaggagcactggccaaaaagccttcagctggcactagataccacaggatattaccgtgcaagtgaactgtgggatataagagaagataatgccaaagatgttgacagtgaaatgacagatgcctctgaggactgccttgaagccagtatgacatattctgaagaagcagaacctgatgataatctcagtgaaaatcttggttcagctgcagaaggaattggcaaacctccacctgtctatgaaacaaagaaggctcggagctaccagattgaacttgcacagcctgctatcaatgggaaaaatgccttaatatgtgcccctactggatctggaaaaactttcgtctcaattctgatttgcgaacaccatttccaaaacatgcctgcaggacgaaaggcgaaagttgtctttcttgcaactaaagtcccagtgtatgaacagcagaaaaatgtcttcaagcatcattttgaaagacaaggatactctgttcaaggaattagtggtgaaaatttttcaaatgtctctgtagaaaaagttatagaggatagtgacatcatcgtagtgacaccccagatcctggtgaatagcttcgaggatgggacccttacctcgctctccattttcactctgatgatattcgatgagtgccacaacactacaggcaaccacccttacaatgtgttaatgaccaggtatctggagcagaaatttaactctgcaagtcagctgccacagattttaggtttgactgcttctgttggagttggtaatgccaagaacattgaggaaacaatagagcacatctgtagtctctgctcctaccttgacatacaggccatatccactgtcagagagaacatacaagaactgcaaaggttcatgaacaagccagaaatagatgtcagattggttaagaggcgaattcacaatccctttgcagccattatctcaaatttgatgtccgagacagaggcactgatgaggacaatttactcagtggatactctctcccaaaacagcaagaaagatttcggaacacagaactatgaacactggatagttgtcactcagaggaaatgcagactgctgcaactagaagacaaagaggaggagagcaggatatgtagagcccttttcatttgcactgaacacctgcggaaatacaatgatgccctcatcatcagtgaagatgcccgcatcatagatgctttatcctacctgaccgagtttttcacaaatgtcaagaatggaccatacacagaattagagcagcacctgacggccaaatttcaagagaaagaaccagaactgattgccctttcaaaagacgaaacaaatgagaatcctaaactggaagagcttgtctgcatcctggatgatgcgtaccgctataacccacagactcgcactcttctctttgctaagacaagagctttagtatccgctttgaagaagtgtatggaggaaaaccctattcttaactacataaagccaggtgttttgatggggcgcggaagaagagatcaaacaacaggtatgaccctcccaagccagaagggtgtgctggatgcgttcaaaaccagcaaggacaacaggctgctcattgctacatctgttgctgatgaaggcattgatattgtccagtgcaaccttgttgtgctctatgaatactccggtaacgtgaccaaaatgatccaagtcagaggtcgtggaagggcagcaggcagcaagtgcatccttgtgacaagcaaaacagaagtggttgagaatgaaaaatgcaaccgttataaggaagaaatgatgaataaagctgttgaaaagatccagaaatgggatgaagaaacatttgcaaaaaagatacataatctgcaaatgaaggaaagggtgttacgagattccaggaggaaagaaataaaacctaaagtagtggaaggccaaaagaacctcctgtgtggaaaatgcaaagcatatgcctgcagtacagatgacatcaggattataaaggactcccatcacattgtcctaggagaagcattcaaggagcgttatacaacaaagcctcataagaaaccaatgcagtttgatggttttgagaaaaaaagcaagatgtattgccgaaataataattgccagcatgattggggaatcacagtgaagtacctgacatttgacaatctaccagtgatcaaaatcaaaagcttcgtaatggagagtactgcaactggaacacaaatggactttcagaaatggaaaagcattaattcttctttgaagaattttgatgttgaagaaatgtccaacttgtacccaccattttagtgcatctattactctcctacactaatgtgaaaaaggagatgatctcctatttccttgtataaaaaatacttgttctttatactgtgtgtgtgctaggtacctcaatggcatctgtctagataacaaaagtctggtatggccaccaaaaaaaaacacacaaaagctatcttattaagtgtttcaccagaacttcatgaagtaagtatggaccaacatatgcctaagtatctagatcttaatctttactgattttattaaaatggtagcatttttctatcatggcatatttgtcagcttgctacagctgaagttgcagaatggatacctttctatgtgttgcttaccttaatgagctgcaagggagacaagacagtgccgtctaagataggtgacggcttaatttagatggtgattaccctgatgttttgcctcaggggttggtatttgaaatctctcacaatgttgatattaattctataggtgccctgtcagctgaattggaatcagaactgagttaaaacattagagtgtttaaatcccttctcctcacctagatcccagacttctctaagaaaaaaacagaagttgctacagatgcaattatgaatggtgatgttaaaactgtaaattatagagcacttgattctacatgataccttcaaagagcataatcttcaaagtttttggaactaagatgaaatatagtttactttctacatttagtagtgcagtaagatattaacatagaggagttatgcatttaattatacgcttgaactctgaagcagaaagcgcttcaaatgataagctaagagtgaaaaatgttttgtaaactacgtaattcctagtcagtgagacttgggatggtcttttaaagcgttgtcccaggccatt

SEQ ID NO: 31 - DNA, 2784 bp, Taeniopygia guttata
atgacggcggaggagaagcagaacttgcggttctacaggcgctacatagagaggagcctaaaccccgtgtacatcctcagcaacatgacggaatggctgtccgatgagatggaggagagagtccggaaggaggaggagaagggagtgacggcggccgccgcgctgttcctggatgccgtgctgctgctggaggcggagggctggttccggggcttccaggatgctctggctgcagcaggttacactggactggcagaagcaattgaaaactgggactttggtaaactggaaaaactggagctgcacaggcagctgttgaagcggatagaatcaacaatgctggaaattgatcctgtagcaatcatgccatacataaacacatgcctgatagagagggaatgtgatgagatcctgcagatcagtgaatacagaagcaaagcggctgggataactaaactcattgaatgcctctgtcgctcggataaggaaaactggcccaaaagtcttcagctggcattggatagtgcaggatattacaatgcaagtgaactgtggaatataagagaagataatggcaaagatgttgatggtgaaatgacagatgcctctgagaataactttgaaaccataatgacattttctgaagaagcagaatgtgataatctgagcaaaaatctctcttcagtttcagaatgcatctgtgagtcttcatctgtttatgaaccaaagaaagctcggagctaccagattgagcttgcacagcctgctattgatgggaaaaacacattgatttgtgcccccacaggatctggaaaaacttttgtggctgttctgatttgtgaacatcatttccaaaacattccctcaggacgaaaggcaaaagttgtcttccttgcaaccaaaatgccagtgtatgagcaacagaaaaatgtattcaggcagcattttgaaagaagtggatactttgttcaaggaatttgtggtgaaacagttgcaaatatctctgtagaaaatgttatacaggacagtgacatcattgtgctaacgccccagattcttgtgaatatcatggataaagggatccttagctccctttccatcttcactctgatgatatttgatgagtgccacaacactgcaggcaaccacccttataatgtgttgatgaccagatacctggatcaaaaatttgactcctctgcaaaacagctgcctcagattgtaggtttaactgcttctgtcggagttggtaatgctaagagcactaaagaaactgtagagcacatctgtaccctctgctcctaccttgacatacaggccatatccactgtcagagagaacaaacaagacctgcagaggtttgcaaacaagccagaaatacatatcagatgggttaaaatgcgagctcagaatcgttttgcagacattatctcaggtctgatgtctgagacagaggttttgatgaggaagatttactcagtggataccatctcccagatcaacaagatttactttggaacacagagatatgaacattggatagtttccactcagaagaaatgcagactattgcaactggaagataaggagaaggagagcagtatttgtagagaccttttcatttgtactgaacacttgcggaaattcaacgatgctctcattatcagtgaagatgctcgcatcgaagatgctttagcctatctaaatgaatttttcacaaatgtaaaaaacggaccatatacagagttagagaagcagctgacagagaaatttcaagagatagaacaagagctgactgccctttcaaaagatgagtcaaatgagaatccaaagttggaagagcttgcttgcatcctggatgaagcataccactataacccacagactcgcactattctctttgccaagacaagagccttagtagctgctttgaagaagtgggtagaagcaaaccctcttcttagccacataaagccagatgtgatgatgggtaagggaagaagagatcgtactgtaggtatgaccctgccaatgcagaagggtgtcctggatgcattcagaaatgacaaagacatcagactgctaattgctacatctgttgctgacgaaggcattgatattactgagtgcaaccttgtggtgctctatgaatacttcggtaatgtcaccaaaatgatccaagtcagaggtcgtggaagggcaaaagacagcaagtgcatccttgtgacaagcaaaacggaagtggttgagaatgaaaaactaaacacttacaaggaagaaatgatgaatgcagctattgaaaagctacagaactgggatgaaacaacatttgcaagaaagatacgtggcctccaaatgaaggaaaaaatgctaagagattccaggaagaatgaaacaaaacatgaactagtggaagggaaaaaaaaactttactgtggaaaatgcaaagcatatgcctgcagtactgatgacatcagaattataaagggatctcatcacattgtcttaggtaacgcattccaggagcgttatacaacaaagccccacaggaaaccggttcagtttgatgattttgtgaaaaaaagcaagatgcattgccgaaataccgagtgccagcatgactgggggatcatagtgaagtacaagatatttgataatctacccgtgatcaaaatcagaagctttgtactagaggatgttgaaagtgggtcacaaatggattttcagaaatggagaagtattaatttgtctttgaagaattttgatgaagaaacatgcagctga

SEQ ID NO: 32 - protein, 459 aa, Anas platyrhynchos
TKDHLDKKKRASEPGKVIVLVNKVPLVEQHLRKEFNPFLKRWYQVIGLSGDSQLKISFPEVVRRYDVIISTAQILENSLLNATEEDEEGVRLSDFSLIIIDECHHTQKEGVYNNIMRRYIKEKMKNRKLAKENKPLIPQPQILGLTASPGVGGANSNQKAEEHILKICANLDARRIMTVEEHASQLKNQVKEPFKKTVIADDKRKDPFRERIIEIMTDIQKYCKLYPKSEFGSQPYEQWVIREEKKAAKEEKRKERVCAEHLKKYNDALQINDTIRMVDAYNHLNNFYKEEKSKKTIGSDDDEPAVSKQDETDEFLLDLFHAKKKQLKELARKPEYENEKLVKLRNTLMEEFTKTKEPRGIIFTKTRQSAFALFQWIKDNPKFEEVGIKAHYLIGAGHNSETKPMTQNEQREVIDKFRGGSVNLLIATTVAEEGLDIKECNIVIRYGLVTNEIAMLQAR

SEQ ID NO: 33 - protein, 459 aa, Gallus gallus
TKDHLDKKRKASEQGKVIVLVNKVPLVEQHLRKEFNPFLKRWYQVIGLSGDSELKISFPEVVKRYDVIICTAQILENSLLNATEEDESVRLSDFSLIIIDECHHTQKEGVYNNIMRRYLKEKIKNRKQAKENKPLIPQPQILGLTASPGVGGARSNSKAEEHILKICANLDACRIMTVKEHASQLKNQVKEPFKKTVIADDKRRDPFRERIIEIMQDIQKYCQLYPKSEFGSQPYEQWVIREERRAAKEEKRKERVCAEHLKKYNDALQINDTIRMVDAYNHLNNFYKELKRRKTAESDDDEEPLVSKQDETDEFLMRLFHAKKKQLKELARKPEYDNEKLMKLRNTLMEEFTKTEEPRGIIFTKTRQSALALYHWIMDNPKFEEVGIKAHFLIGAGHNSETKPMTQNEQREVIDKFRGGSINLLIATTVAEEGLDIKECNIVIRYGLVTNEIAMVQAR

(19) United States
(12) Patent Application Publication
(10) Pub. No.: US 2011/0247091 A1
(43) Pub. Date: Oct. 6, 2011

(54) Transgenic Cells and Chickens Expressing RIG-I

(75) Inventors: Katharine Edens Magor, Edmonton (CA); Megan Rani Winnifred Barber, Edmonton (CA); Jerry Ronald Aldridge, JR., Cordova, TN (US); Robert G. Webster, Memphis, TN (US)

(73) Assignee: THE GOVERNORS OF THE UNIVERSITY OF ALBERTA

(21) Appl. No.: 13/072,190

(22) Filed: Mar. 25, 2011

Related U.S. Application Data
(60) Provisional application No. 61/318,034, filed on Mar. 26, 2010.

(51) Int. Cl. A01K 67/027; C12N 5/10; C12N 15/873

(52) U.S. Cl. 800/19; 435/349; 800/25

(57) ABSTRACT

The present disclosure provides a transgenic cell or chicken having increased resistance to RNA viral infection, wherein the chicken cell expresses a foreign RIG-I gene. Also provided are methods for reducing RNA virus replication in a chicken cell and methods for producing the transgenic cell or chicken with increased RNA viral resistance.

### RELATED APPLICATION

This application claims the benefit under 35 USC §119(e) from U.S. Provisional patent application Ser. No. 61/318,034, filed Mar. 26, 2010, which is incorporated herein by reference in its entirety.

### STATEMENT REGARDING FEDERALLY SPONSORED RESEARCH OR DEVELOPMENT

This invention was made in part with U.S. Government support under Contract HHSN266200700005C awarded by the National Institute of Allergy and Infectious Diseases, National Institutes of Health. The U.S. Government has certain rights in this invention.

### FIELD

The disclosure relates to transgenic cells and chickens that express RIG-I and methods of producing transgenic cells and chickens with increased viral resistance.

### BACKGROUND

Ducks are the reservoir of influenza viruses, and are involved in their propagation and evolution. Recently, influenza strains have emerged that are fatal to poultry, but often result in asymptomatic infection in ducks. Co-adaptation of ducks and wild aquatic birds with influenza, has resulted in generally asymptomatic infections that ensure perpetuation of all influenza subtypes in the natural reservoir (Webster et al. 1992). Chickens rapidly succumb to highly pathogenic avian influenza (HPAI) infections, while ducks are naturally resistant. Some strains of H5N1 cause 100% mortality in chickens, but often result in asymptomatic infection in ducks, making them a ‘Trojan horse’ in the spread of the virus (Kim et al. 2009; Hulse-Post et al. (2005); Hulse-Post et al. 2007)). The molecular basis of the successful response of ducks to influenza infection is unresolved, but given that influenza virus causes an acute infection, the innate immune response is critical. A successful innate immune response to influenza infection involves a robust, yet transient upregulation of interferon-stimulated antiviral genes, particularly those downstream of the RIG-I pattern recognition receptor. RIG-I is a cytoplasmic RNA sensor, and triggering by influenza virus leads to production of IFN-β and expression of downstream interferon-stimulated antiviral genes (Loo et al. 2008). Interference in RIG-I expression and downstream genes is a hallmark of a lethal influenza virus infection (Kobasa et al. 2007) and RNA viruses are more virulent and replicate to higher levels in mice lacking RIG-I (Kato et al. 2006).

### SUMMARY

The present inventors have shown a difference in susceptibility to influenza due to Retinoic Acid Inducible Gene-I (RIG-I), a pattern recognition receptor indispensable for influenza detection and initiation of the interferon response. The present disclosure shows that RIG-I is absent in chickens, but functional in ducks and provides evidence for the role of RIG-I in protection against influenza infection in the natural host. Expression of duck RIG-I in chicken cells rescues detection of RIG-I ligand, increases the interferon response and reduces influenza replication. RIG-I is highly upregulated in duck lung tissue infected with H5N1 A/Vietnam/1203/04 (VN1203), a strain that is lethal in chickens.

Accordingly, the present invention provides a method of reducing RNA virus replication in a chicken cell comprising expressing a recombinant nucleic acid molecule encoding a RIG-I protein or functional variant or fragment thereof in the chicken cell. In one embodiment, RIG-I is an avian RIG-I, such as duck RIG-I, zebra finch or pigeon RIG-I. In an embodiment, the duck RIG-I comprises the amino acid sequence as shown in SEQ ID NO: 1. In another embodiment, the zebra finch RIG-I comprises the amino acid sequence as shown in SEQ ID NO:2.

The disclosure also includes a chicken cell transformed with a recombinant nucleic acid molecule encoding a RIG-I protein or functional variant or fragment thereof.

The present disclosure also provides a transgenic chicken with resistance to RNA viruses comprising a recombinant nucleic acid molecule encoding a RIG-I protein or functional variant or fragment thereof. In one embodiment, RIG-I is an avian RIG-I, such as duck RIG-I, zebra finch RIG-I or pigeon RIG-I. In an embodiment, the duck RIG-I comprises the amino acid sequence as shown in SEQ ID NO:1. In another embodiment, the zebra finch RIG-I comprises the amino acid sequence as shown in SEQ ID NO:2.

In another embodiment, the recombinant nucleic acid molecule is a plasmid or viral vector. In an embodiment, the viral vector is a retroviral vector, an adenoviral vector or an adeno-associated viral vector. In another embodiment, the retroviral vector is a replication defective retrovirus vector.

In yet another embodiment, the recombinant nucleic acid molecule is integrated into the genome of said chicken or is expressed in its germ cells and somatic cells.

Also provided herein is a transgenic cell or cell line obtained from the transgenic chicken disclosed herein.

Further provided are methods of producing a transgenic chicken with increased resistance to RNA viruses comprising a foreign RIG-I gene. In one embodiment, the RNA virus is a single stranded negative RNA virus such as influenza, optionally H5N1, or Newcastle Disease Virus (NDV). In one embodiment, the vector is a replication defective retroviral vector.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows amino acid alignment of duck, zebra finch and human RIG-I. Alignment of duck RIG-I (SEQ ID NO:1, accession number EU363349), zebrafinch (SEQ ID NO:2, accession number XM_{—}002194524) and human RIG-I (SEQ ID NO:3, accession number AF038963) was performed using the Clustal W program and edited with BOXSHADE. Black shading indicates amino acid identity, grey shading indicates similarity (50% threshold). Plus signs indicate residues involved in polyubiquitination, asterisks indicate residues involved in ligand binding. The ATP binding motif is boxed.

FIG. 2 shows RIG-I is present in ducks and pigeons, but absent in chickens. (A) Hybridization of a multiple exon duck RIG-I probe to HindIII and XbaI-digested genomic DNA from four White Pekin ducks and two White Leghorn chickens. (B) Hybridization of a single axon duck RIG-I probe to PstI, NdeI and SacI digested genomic DNA from duck, chicken and pigeon. (C) Hybridization of a single axon duck MDA5 probe to same blot.

FIG. 3 shows duck RIG-I rescues detection of 5′ppp RNA and induction of an antiviral response in DF-1 chicken embryonic fibroblast cells. (A) IFN-β promoter activity in DF-1 cells following ligand stimulation compared to mock-treated cells, shown as mean fold induction (+/−s.d.). DF-1 cells were co-transfected with pcDNA-RIG or empty vector, and a chicken IFN-β promoter construct. 30 hours post-transfection, cells were transfected with 800 ng 5′ppp RNA or CIAP-treated 5′ppp RNA or 25 μg/mL poly(I:C). Results are representative of 3 independent experiments and were analysed using a single-factor ANOVA and Tukey's posthoc test (different letters P<0.05). (B) RIG-I transfection increases DF-1 cell expression of chicken IFN-β and the interferon stimulated genes Mx1 and PKR, and decreases influenza matrix gene expression. Results are representative of 3 independent experiments and error bars show RQ_{Min/Max} at a 95% confidence level and represent standard error (n=3). (C) Influenza virus titre is significantly reduced in RIG-I transfected DF-1 cells. 24 hours post-transfection, cells were infected with H5N2. After 15 hours, titre was determined by plaque assay from triplicate wells and results were analyzed with the two-tailed Student's t test (n=3, P=0.002).

FIG. 4 shows RIG-I is significantly upregulated in duck lung infected with HPAI but not LPAI. Lung and intestinal RNA was extracted d1 or d3 post infection (PI) and analyzed by qRT-PCR for RIG-I expression as compared to mock-infected animals. (A) Fold expression of RIG-I mRNA in duck lung d1 PI with H5N2 or H5N1. (B) Fold expression of RIG-I mRNA in duck lung d3 PI. (C) Fold expression of RIG-I mRNA in duck intestine d1 PI. (D) Fold expression of RIG-I mRNA in duck intestine d3 PI. Error bars show RQ_{Min/Max} at a 95% confidence level and represent standard error (n=4).

FIG. 5 shows duck (SEQ ID NO:32, accession number GU936632) and chicken (SEQ ID NO:33, accession number XM422031) MDA5 are highly conserved. Alignment of partial duck MDA5 and chicken MDA5 was performed using the Clustal W program and edited with BOXSHADE. Black shading indicates amino acid identity, grey shading indicates similarity (50% threshold).

FIG. 6 shows the duck RIG-1 nucleic acid sequence (SEQ ID NO:30, accession number EU363349).

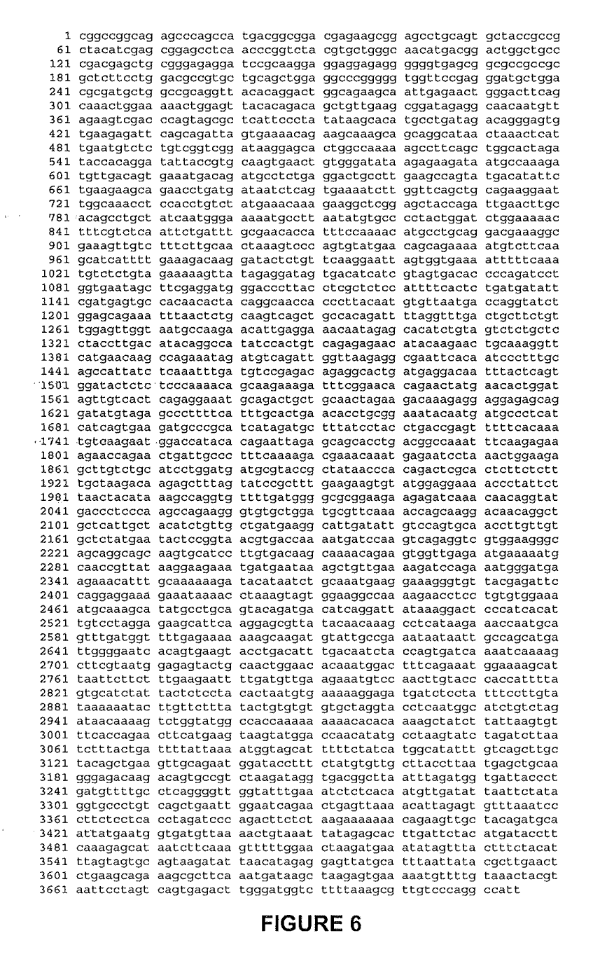

FIG. 7 shows the zebra finch RIG-1 nucleic acid sequence (SEQ ID NO:31, accession number XM_{—}002194524).

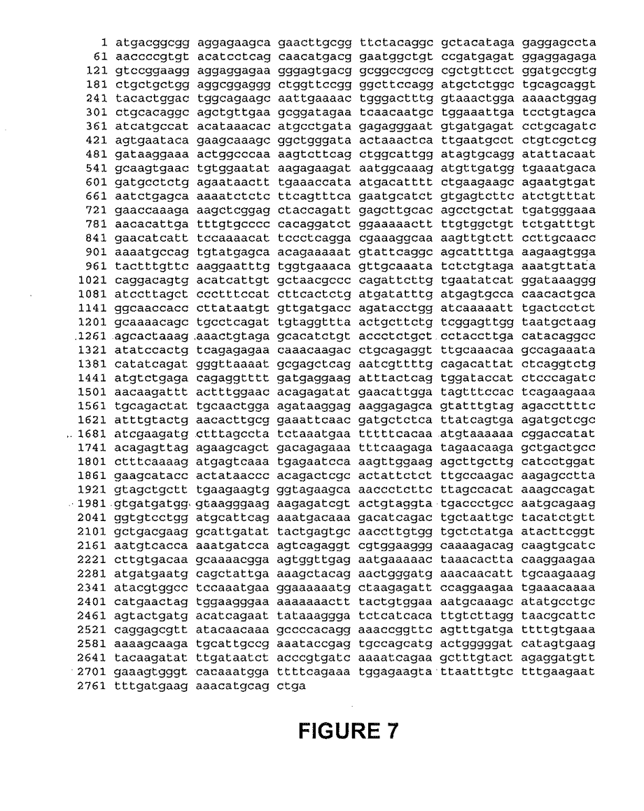

### DETAILED DESCRIPTION

The present inventors have shown that the chicken immune response is impaired in their ability to recognize RNA viral infection because they are missing the receptor, retinoic acid-inducible gene 1 (RIG-I) that initiates the innate antiviral response. Accordingly, production of transgenic chickens that express RIG-I will render the chickens capable of clearing the infection by boosting their natural immune response through the RIG-I receptor that recognizes the RNA virus infection. Further, RIG-I expression would be expected to increase efficacy of a vaccine.

Accordingly, the present invention provides a method of reducing RNA virus replication in a chicken cell comprising expressing a recombinant nucleic acid molecule encoding a RIG-I protein or functional variant or fragment thereof in the chicken cell.

The term “RNA virus” as used herein means a virus that has ribonucleic acid (RNA) as its genetic material, including without limitation, single stranded viruses such as influenza and Newcastle disease virus. In one embodiment, the influenza virus is H5N1.

The term “RIG-I” or “retinoic acid-inducible gene 1” as used herein refers to a RIG-I protein from any species or source and includes the full-length protein as well as variants, fragments or portions thereof. Duck RIG-I protein has the amino acid sequence as shown in SEQ ID NO:1 and the nucleic acid sequence as shown in SEQ ID NO:30. Zebra finch RIG-I comprises the amino acid sequence as shown in SEQ ID NO:2 and the nucleic acid sequence as shown in SEQ ID NO:31. Human RIG-I comprises the amino acid sequence as shown in SEQ ID NO:3.

In one embodiment, the RIG-I protein, variant or functional fragment thereof is an avian RIG-I protein or is encoded by an avian RIG-I gene. The avian RIG-I may be derived from any bird that contains a RIG-I gene. A person skilled in the art would readily be able to determine whether a particular type of bird has a RIG-I gene. For example, the bird's DNA or RNA can be isolated and a RIG-I probe, such as that disclosed in the present Examples, can be used to detect and isolate a RIG-I homolog, for example, pigeon RIG-1, or proteins can be isolated from the bird and an antibody to RIG-I can be used to detect and isolate a RIG-I homolog. In one embodiment, the avian RIG-I is a duck RIG-I, such as duck RIG-I having the amino acid sequence as shown in SEQ ID NO:1. In another embodiment, the avian RIG-I is a pigeon RIG-I. In yet another embodiment, the avian RIG-I is a zebra finch RIG-I, optionally having the amino acid sequence as shown in SEQ ID NO:2.

The term “variant” as used herein includes modifications, substitutions, additions, derivatives, analogs, fragments or chemical equivalents of the RIG-I amino acid sequence that perform substantially the same function in substantially the same way. A functional variant of RIG-I would retain the activity of recognizing RNA viruses and/or inducing an innate immune response.

The term “RIG-I protein fragment” as used herein means a portion of the RIG-I peptide that contains at least 10%, 20%, 30%, 40%, 50%, 60%, 70%, 80%, 90%, 95%, or more of the entire length of the RIG-I polypeptide. A functional fragment of RIG-I would retain the activity of recognizing RNA viruses and/or inducing an innate immune response.

The term “homolog” means those amino acid or nucleic acid sequences which have slight or inconsequential sequence variations from the RIG-I sequence, i.e., the sequences function in substantially the same manner. The variations may be attributable to local mutations or structural modifications. Sequences having substantial homology include nucleic acid sequences having at least 50%, at least 65%, at least 85%, or at least 90-95% identity with the RIG-I sequence. Sequence identity can be calculated according to methods known in the art. Nucleic acid sequence identity is most preferably assessed by the algorithm of BLAST version 2.1 advanced search. BLAST is a series of programs that are available online at http://www.ncbi.nlm.nih.gov/BLAST. The advanced blast search (http://www.ncbi.nlm.nih.gov/blast/blast.cgi?Jform=1) is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_{—}141; Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs,” Nucleic Acids Res. 25:33893402; Zhang, J. & Madden, T. L. (1997) “PowerBLAST: A new network BLAST application for interactive or automated sequence analysis and annotation.” Genome Res. 7:649656.

The term “analog” means an amino acid or nucleic acid sequence which has been modified wherein the modification does not alter the utility of the sequence (e.g. for recognizing RNA viruses or for inducing innate immunity) as described herein. The modified sequence or analog may have improved properties over the RIG-I sequence. One example of a nucleic acid modification to prepare an analog is to replace one of the naturally occurring bases (i.e. adenine, guanine, cytosine or thymidine) of the sequence with another naturally occurring base or modified base such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules encoding RIG-I. For example, the nucleic acid sequences may contain phosphorothioates, phosphotriesters, methyl phosphonates, and phosphorodithioates.

The disclosure also includes nucleic acids having sequences that hybridize to the RIG-I sequence or a fragment thereof and encode a protein that maintains the property of innate immune activation and viral recognition. The term “sequence that hybridizes” means a nucleic acid sequence that can hybridize to a RIG-I sequence under stringent hybridization conditions. Appropriate “stringent hybridization conditions” which promote DNA hybridization are known to those skilled in the art, or may be found in Current Protocols in Molecular Biology, John Wiley & Sons, N.Y, (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.−16.6(Log 10+[Na+])+0.41 (%(G+C)−600/l). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations stringent hybridization conditions shall be defined as: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C.

The RIG-I protein may be modified to contain amino acid substitutions, insertions and/or deletions that do not alter the properties of the protein. Conserved amino acid substitutions involve replacing one or more amino acids of the protein with amino acids of similar charge, size, and/or hydrophobicity characteristics. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. When only conserved substitutions are made the resulting analog should be functionally equivalent to the RIG-I protein. Non-conserved substitutions involve replacing one or more amino acids of the RIG-I protein with one or more amino acids which possess dissimilar charge, size, and/or hydrophobicity characteristics.

The term “nucleic acid” or “nucleic acid molecule” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

In an embodiment, the recombinant nucleic acid is a recombinant expression vector, such as a plasmid or viral vector. Plasmid and viral vectors useful in creating transgenic chickens are readily known in the art. The expression vectors are “suitable for transformation of a host chicken cell”, which means that the expression vectors contain a nucleic acid molecule and regulatory sequences selected on the basis of the host chicken cells to be used for expression, which is operatively linked to the nucleic acid molecule. Operatively linked is intended to mean that the nucleic acid is linked to regulatory sequences in a manner which allows expression of the nucleic acid.

The disclosure therefore includes a recombinant expression vector containing a nucleic acid molecule disclosed herein, or a fragment thereof, and the necessary regulatory sequences for the transcription and translation of the inserted protein-sequence. Selection of appropriate regulatory sequences is dependent on the host chicken cell, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors may also contain a selectable marker gene which facilitates the selection of host cells transformed or transfected with a recombinant molecule disclosed herein. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of the recombinant expression vectors disclosed herein and in particular to determine the effect of a mutation on expression and phenotype. It will be appreciated that selectable markers can be introduced on a separate vector from the nucleic acid of interest.

In one embodiment, a leaky promoter, such as a cytomegalovirus (CMV) promoter is operatively linked to the transgene so that RIG-I is expressed in many if not all of the tissues of the transgenic chicken.

In an embodiment, the vector is a viral vector, optionally a retroviral, adenoviral or adeno-associated viral vector. Such vectors are able to deliver the recombinant nucleic acid into the cells and the nucleic acids are then stably integrated into the chromosomal DNA of the host.

In an embodiment, the retroviral vector is a replication defective retrovirus vector. The term “replication defective retrovirus vector” as used herein refers to a retrovirus that is capable of insertion of a foreign gene into cells in an initial round of infection but where the vectors would not permit re-infection of the cells after the initial round of infection. For example, a replication defective retrovirus may have the retroviral coding sequences, such as gag, pol and env, replaced by other nucleic acids or deleted, which render the virus unable to replicate. Replication defective virus vectors have been described in Bosselman et al. Mol. Cell. Biol. 7: 1797-1806 (1987); U.S. Pat. No. 5,162,215; Cone and Mulligan, PNAS USA 81:6349-6353 (1984); Mann et al. Cell 33:153-159 (1983); Miller and Buttimore, Mal. Cell. Biol. 6:2895-2902 (1986), Watanabe and Temin, Mol. Cell. Biol. 3:2441-249 (1983); Stoker and Bissell, J. Virol. 62; 1008-1015 (1988), U.S. Pat. No. 4,650,764 and Emerman and Temin, Cell, 39:459-467 (1984), all of which are herein incorporated by reference. For example, replication defective retroviral vectors include, without limitation, replication defective retrovirus vectors derived from Moloney murine leukemia virus, Moloney murine sarcoma virus, reticuloendotheliosis virus type A and Avian leukemia virus.

In yet another embodiment, an adenovirus-derived vector is used. The adenovirus vector can be modified such that its ability to replicate in a normal lytic viral life cycle is inactivated (see for example, Berkner et al., 1988, BioTechniques 6:616; Rosenfeld et al., 1991, Science 252:431-434 and Rosenfeld et al., 1992, Cell 68:143-155; all of which are incorporated by reference). Introduced adenoviral vectors are not integrated into the genome of the host cell but instead remain episomal.

In yet another embodiment, an adeno-associated virus is used. Such viruses can be packaged and can integrate. See for example, Tratschin et al. 1985, Mol. Cell. Biol. 4:2072-2081; Hermonat et al. PNAS, 1984, 81:6466-6470; Wondisford et al. 1988, Mol. Endocrinol. 2:32-39; Tratschin et al. 1984, J. Virol 51:611-619 and Flotte et al. 1993, J. Biol. Chem. 268:3781-3790, all of which are incorporated herein by reference.

Non-viral expression vectors can also be used. For example, a RIG-I encoding nucleic acid can be entrapped in liposomes and targeted to cells (see WO 91/06309 and EP-A-43075, which are incorporated herein by reference).

Recombinant expression vectors can be introduced into host cells to produce a transformed host cell. The term “transformed host cell” is intended to include cells that are capable of being transformed or transfected with a recombinant expression vector of the disclosure. The terms “transduced”, “transformed with”, “transfected with”, “transformation” and “transfection” are intended to encompass introduction of nucleic acid (e.g. a vector or naked RNA or DNA) into a cell by one of many possible techniques known in the art. Nucleic acid can be introduced into eukaryotic cells via conventional techniques such as calcium phosphate or calcium chloride co-precipitation, DEAE-dextran mediated transfection, lipofectin, electroporation, microinjection, RNA transfer, DNA transfer, artificial chromosomes, viral vectors and any emerging gene transfer technologies. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

The disclosure also includes a chicken cell transformed with a recombinant nucleic acid molecule encoding a RIG-I protein or functional variant or fragment thereof.

The present disclosure also provides a transgenic chicken comprising a recombinant nucleic acid molecule encoding a RIG-I protein or functional variant or functional fragment thereof.

The term “transgenic chicken” as used herein refers to a chicken that contains a heterologous gene in at least a portion of its cells. The nucleic acid is introduced into the cell, directly or indirectly by introduction into a precursor of the cell, by genetic manipulation, including without limitation, sperm-mediated or restriction enzyme mediated integration, microinjection or viral infection. In one embodiment, the recombinant nucleic acid is stably integrated into the genome of the chicken. In another embodiment, the recombinant nucleic acid is expressed in its germ cells and/or somatic cells.

The transgenic chickens described herein have increased resistance to RNA viruses including, without limitation, negative single stranded viruses such as influenza and Newcastle disease virus. In an embodiment, the transgenic chicken is resistant to influenza. In one embodiment, the influenza virus is H5N1.

In another aspect, the present disclosure provides a transgenic cell or cell line obtained from the transgenic chicken disclosed herein.

In yet another aspect, the present disclosure provides a method of producing a transgenic chicken with increased resistance to RNA viruses comprising a foreign RIG-I gene.

For example, transgenic chickens may be produced by using a retrovirus vector, embryonic stem cells, primordial germ cells or spermatozoa. Methods of producing the transgenic chickens having a RIG-I gene include, without limitation, introducing the transgene to the chicken using, a viral or a non-viral vector; sperm-mediated gene transfer; restriction enzyme-mediated integration; nuclear transfer; ovum transfer and the like. Transgenic chickens produced by the present methods will be able to lay eggs and/or produce offspring containing the RIG-I heterologous protein.

Methods of making transgenic chickens are known in the art (see for example, U.S. Pat. No. 5,162,215 to Bosselman et al., US 2002/0108132 to Rapp and US 2009/0158449 to Nakaishi et al., all of which are incorporated herein by reference).

Bosselman et al. describe introduction of a replication defective retroviral vector into pluripotent stem cells of a chick embryo. Briefly, an opening, for example an opening about 5 mm in diameter, is made in a laid chicken egg which is not more than two days old. Typically the opening in the laid chicken egg is made using a drilling tool with an abrasive rotating tip to drill a hole in the eggshell but maintaining the underlying shell membrane intact. The membrane is cut out by a scalpel to expose the embryo which can be visualized with an optical dissecting microscope having 6× to 50× magnification. The solution comprising the DNA or vector can be any suitable medium, such as tissue culture medium, which is then microinjected into an area beneath and around the blastoderm. Typically 5-20 μl of solution is microinjected. Microinjection is carried out using a micromanipulator and a very small diameter needle, optionally a glass needle with 40 to 60 μm outer diameter. The egg is then sealed with shell membrane and a sealing material, such as glue, cement or paraffin. The sealed microinjected egg is incubated at 37° C. to allow development of the embryo until the egg hatches. The hatched chickens are then genotyped, for example, by analysis of blood DNA and positive chickens are bred to produce progeny transgenic chickens.

Accordingly, in one embodiment, the present disclosure provides a method of producing a transgenic chicken with increased resistance to RNA virus infection, comprising:

inserting into a chicken embryo a recombinant nucleic acid molecule comprising a foreign RIG-I gene; and

allowing the embryo to hatch.

In an embodiment, the recombinant nucleic acid molecule is a viral vector. In one embodiment, the viral vector is a retroviral vector, an adenoviral vector or an adeno-associated viral vector. In another embodiment, the retroviral vector is a replication defective retroviral vector.

Alternatively, the DNA or vector comprising the DNA may be injected into chicken embryonic stem cells or primordial germ cells, which are then injected into recipient embryos. See for example, US 2003/0115622 to Ponce de Leon et al., US 2006/0075513 to Ivarie et al. and US 2006/0206952 to Van de Lavoir and Leighton, all of which are incorporated herein by reference. Briefly, Ponce de Leon et al. describe excising blastodermal cells containing presumptive primordial germ cells (PGCs) from donor eggs, typically at stage XII to XIV. The PGCs are cultured in suitable culture medium containing factors to promote proliferation, such as LIF, bFGF, SCF and IGF to produce embryonic germ cells, which are typically produced after 28 days of culture. The cultured PGCs are introduced with a transgene by any suitable means, such as lipofection, transfection, microinjection, transformation, etc. The transgenic PGCs are then introduced into the dorsal aorta of recipient embryos, typically around stage 13-14. Similar to the above protocol from Bosselman et al., a small opening in shell is made, typically with forceps, and the PGCs are injected using a micropipette. Typically 200 donor PGCs are injected. After injection, the opening is sealed as described herein. The transfected donor cells become incorporated into the recipient embryos, some of which will produce chickens with the transgene in the germline.

Alternatively, WO87/05325 to Rottman and Höfer describe transferring material into sperm or egg cells by using liposomes containing the recombinant DNA or vector. The transgenic donor egg can then be transferred to the oviduct of a recipient hen (see Tanaka et al. 1994, J. Reprod. And Fertility, 100:447-449, incorporated herein by reference) or male germ cells can be transferred to a recipient testis. Restriction enzyme mediated integration is an alternative method for creating transgenic sperm (see Shemesh et al. in WO 99/42569, incorporated herein by reference).

Accordingly, in another embodiment, the present disclosure provides a method of producing a transgenic chicken with increased resistance to RNA virus infection, comprising:

inserting into a chicken germline or embryonic stem cell a vector comprising a foreign RIG-I gene;

producing a zygote from said germline or embryonic stem cell;

allowing the zygote to form an embryo in the chicken; and

allowing the embryo to hatch.

The term “germline cell” as used herein refers to a germ cell, such as a primordial germ cell, or sex cell, such as sperm or egg, that have DNA that is passed on to offspring. The term “zygote” as used herein refers to the cell resulting from the union of sperm and egg.

The phrase “producing a zygote from a germline or embryonic stem cell” as used herein includes, without limitation, fertilization of a non-transgenic egg with a transgenic sperm cell or fertilization of a transgenic egg with a non-transgenic sperm cell or microinjection of a transgenic germline cell or embryonic stem cell or culture of cells into a non-transgenic zygote or ovum or sperm transfer.

The term “embryo” as used herein refers to the product of the zygote inside the chicken egg.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Materials and Methods

Identification and cloning of duck RIG-I. A PCR fragment of duck RIG-I was obtained from splenic cDNA using primers 5′-GAT CCC AGC AAT GAG AAT CCT AAA CT-3′ (SEQ ID NO:4) and 5′-CAA TGT CAA TGC CTT CAT CAG C-3′ (SEQ ID NO:5). The complete cDNA sequence was obtained via 5′ and 3′ RACE (Rapid Amplification of cDNA Ends) using the SMART™ RACE cDNA Amplification Kit (Clontech), Duck RIG-I was amplified using primers in the 5′UTR 5′-CGG CCG GCA GAG CCC AGC C-3′ (SEQ ID NO:6) and 3′ UTRs 5′-GTG TAG GAG AGT MT AGA TGC ACT A-3′ (SEQ ID NO:7) using Phusion High-Fidelity DNA Polymerase (New England Biolabs), cloned into pCR 2.1-TOPO (Invitrogen) and completely sequenced.

Plasmids, pcDNA-RIG was obtained by cloning duck RIG-I into the mammalian expression vector pcDNA 3.1 hygro+ (Invitrogen). Duck RIG-I was digested out of pCR 2.1-TOPO (Invitrogen) using SpeI and NotI. RIG-I was then inserted between NheI and NotI sites of pcDNA 3.1 hygro+ (Invitrogen). The chicken IFN-β promoter luciferase reporter (pGL3-chIFNβ) was constructed from White Leghorn chicken genomic DNA using primers with incorporated BgIII and MluI sites that amplified −158 to +14 of the chicken IFN-2 promoter, as previously done (Childs et al. 2007; Sick et al. 1998). The promoter fragment was then inserted between BgIII and MluI sites of the pGL3-basic luciferase reporter vector (Promega Wis.).

In vitro transcription and RNAs. The 21-mer RNA, 5′-pppGGGGCUGACCCUGAAGUUCCC-3′ (SEQ ID NO:8) (Hornung et al. 2006) was transcribed from annealed DNA oligonucleotides 5′ TAA TAC GAC TCA CTA TAG GG-3′ (SEQ ID NO:9) and 5′-GGG AAC TTC AGG GTC AGC CCC TAT AGT GAG TCG TAT TA-3′ (SEQ ID NO:10) containing a T7 promoter site, using the T7 Megashortscript kit (Ambion). In vitro transcription was carried out overnight, followed by DNasel digestion and precipitation. Calf intestinal alkaline phosphatase (CIAP) treatment was carried out for 3 hours using 30 μg of in vitro transcribed RNA, 30 U of CIAP (Invitrogen), 1×CIAP buffer and 200 U RNase Out (Invitrogen). CIAP treated 5′ppp RNA was purified by phenol-chloroform extraction and precipitation. Poly (I:C) (25 mg/mL) was obtained from InVivogen.

Cell culture and Transfections. UMNSAH/DF-1, a spontaneously immortalized chicken embryonic fibroblast cell line derived from East Lansing strain eggs (Himly et al. 1998) was maintained in DMEM +10% FBS. 1.25×10⁵ cells were seeded overnight in 24-well plates. Cells were co-transfected with 150 ng of pcDNA-RIG or empty pcDNA, 150 ng of pGL3-chIFNβ, and 10 ng of the constitutive renilla luciferase reporter phRG-TK (Promega). 30 hours post plasmid-transfection, cells were challenged with 21-mer 5′ppp RNA, CIAP-treated RNA or poly (I:C). Plasmids and RNA ligands were transfected using Lipofectamine 2000 (Invitrogen). Luciferase activity was measured using the Dual-Luciferase Reporter Assay System (Promega) 15 hours post-challenge.

Viruses and Duck Infections. The H5N1 A/Viet Nam/1203/04 HPAI was generated by reverse genetics (Salomon et al. 2006) and H5N2 A/mallard/BC/05 LPAI was isolated by screening of environmental samples. The viruses were propagated in 10-day-old embryonated chicken eggs and handled at St. Jude Children's Research Hospital, with H5N1 handled in biosafety level 3+ facilities approved by the U.S. Department of Agriculture and Centers for Disease Control and Prevention. Outbred White Pekin ducks (Anas platyrhynchos) were purchased from Ideal Poultry (Cameron, Tex.) or Metzer Farms (Gonzales, Calif.) and all animal experiments were approved by the Animal Care and Use Committee of St. Jude and performed in compliance with relevant institutional policies, National Institutes of Health regulations, and the Animal Welfare Act. 10⁶ EID⁵⁰ (50% egg infectious dose) of H5N2 and H5N1 were used to inoculate six-week-old mallards via the natural route, as previously described (5). Ducks were sacrificed and tissues collected at d1 and d3 PI (n=3, except for H5N2 d3 samples (n=2)). Tracheal and cloacal swabs were collected to monitor viral shedding.

Southern hybridization. Genomic DNA (10 μg) was extracted from blood of White Pekin ducks (Anas platyrynchos), White Leghorn chickens (Gallus gallus) and pigeon (Columba livia). Genomic DNA was digested to completion, separated on 0.8% agarose and blotted to Nytran Supercharge (Schleicher & Schuell). DNA was immobilized by UV cross-linking and baking for 3 hours at 80° C. A multiple exon 307 bp RIG-I probe in the helicase domain was amplified using the primers 5′-ACA GGT ATG ACC CTC CCA AGC CAG-3′ (SEQ ID NO:11) and 5′-CAT CCC ATT TCT GGA TCT TTT CAA CAG-3′ (SEQ ID NO:12) from a defined duck RIG-I clone. A 171 bp probe predicted to contain a single exon was constructed using 5′-GTA TGA CCC TCC CAA GCC AGA AGG-3′ (SEQ ID NO:13) and 5′-CTC TGA CTT GGA TCA TTT TGG TCA CG-3′ (SEQ ID NO:14). A 150 bp duck MDA5 probe was constructed with the following primers: 5′-GAG CAA AGG GM GTC ATT GAT AAA TTC C-3′ (SEQ ID NO:15) and 5′-GGC CTG CM CAT AGC AAT TTC ATT-3′ (SEQ ID NO:16). All probes were radiolabelled with ³²P α-dCTP by random priming (Primelt Random Primer Labeling Kit, Stratagene). Blots were hybridized overnight at 42° C. in 50% formamide, 5×Denhardt's solution, 4×SSPE, 5% dextan sulphate, 1% SDS and 100 μg/mL salmon sperm DNA. Washes were carried out at low stringency in 1×SSPE and 0.1% SDS at 52° C. Film was exposed for 3 days.

Real-time quantitative reverse transcription-PCR (qRT-PCR). RNA was extracted using TRIZOL (Invitrogen), followed by purification from the final aqueous phase using the RNeasy Mini Kit (Qiagen). First strand cDNA synthesis was performed using the Superscript III kit (Invitrogen) using OligodT (Invitrogen) and random primers (Invitrogen). To quantify RIG-I gene expression from duck tissues, 50 ng of cDNA was amplified in a 10 μL reaction using the Applied Biosystems 7500 Real Time PCR system (Applied Biosystems). Duck primers and fluorogenic TaqMan FAM/TAMRA (6-carboxyfluorescein/6-carboxytetramethylrhodamine)-labeled hybridization probe mixes were obtained from Applied Biosystems and used with FastStart Universal Probe Master (Rox) (Roche Applied Science). Duck glyceraldehyde-3-phosphate dehydrogenase (GAPDH), was used as an endogenous control. Primer and probe sequences were as follows: Duck RIG-I primers 5′-GTG TAT GGA GGA AAA CCC TAT TTC TTA ACT-3′ (SEQ ID NO:17) and 5′-GGA GGG TCA TAC CTG TTG TTT GAT-3′ (SEQ ID NO:18), and probe 5′-TTC CGC GCC CCA TCA A-3′ (SEQ ID NO:19). Duck GAPDH primers 5-GCC TCT TGC ACC ACC AAC T-3′ (SEQ ID NO:20) and 5′-GGC ATG GAC AGT GGT CAT MG AC-3′ (SEQ ID NO:21) and probe 5′-CAC MT GCC AAA GTT G-3′ (SEQ ID NO:22). Changes in gene expression PI were expressed as a ratio of the level observed in a mock-infected animal. RT-PCR was performed for RIG-I and GAPDH in a singleplex format, with the following cycling conditions: 95° C. for 10 minutes for activation, followed by 40 cycles at 95° C. for 15 s, and 60° C. for 1 min. Quadruplicate cycle threshold C_{T} values were analyzed with SDS software (Applied Biosystems) using the comparative C_{T} (ΔΔC_{T}) method.

To quantify gene expression from transfected DF-1 cells, 20 ng of cDNA was amplified in a 10 μL reaction using the Applied Biosystems 7500 Real Time PCR system (Applied Biosystems). Chicken 28s RNA (endogenous control) and IFN-β probes and primer (Peters et al. 2003) were obtained from Applied Biosystems and used with Taqman Fast Universal PCR Master Mix (Applied Biosystems). For Influenza A matrix gene expression, primers were designed according to CDC recommendations, InfA forward: 5′-GAC CRA TCC TGT CAC CTC TGA C-3′ (SEQ ID NO:23), InfA reverse 5′-AGG GCA TTY TGG ACA AAK CGT CTA-3′ (SEQ ID NO:24) and InfA probe 5′-TGC AGT CCT CGC TCA CTG GGC ACG-3′ (SEQ ID NO:25) (Centers for Disease Control and Prevention). The primers and probes for chicken Mx1 (accession number NM204609) and PKR (accession number NM_{—}204487.1) were designed with Roche's online Universal Probe Library (UPL) system's Assay Design Center. The primers for Mx1 were 5′-GTT TCG GAC ATG GGG AGT AA-3′ (SEQ ID NO:26) and 5′-GCA TAC GAT TTC TTC AAC TTT GG-3′ (SEQ ID NO:27) (UPL probe 80). The primers for PKR were 5′-TGC TTG ACT GGA MG GCT ACT-3′ (SEQ ID NO:28) and 5′-TCA GTC MG AAT AAA CCA TGT GTG-3′ (SEQ ID NO:29) (UPL probe 29). Cycling conditions were 95° C. for 20 seconds for activation, followed by 40 cycles at 95° C. for 3 s, and 60° C. for 30 s. Changes in gene expression were expressed and analyzed as above.

### Results

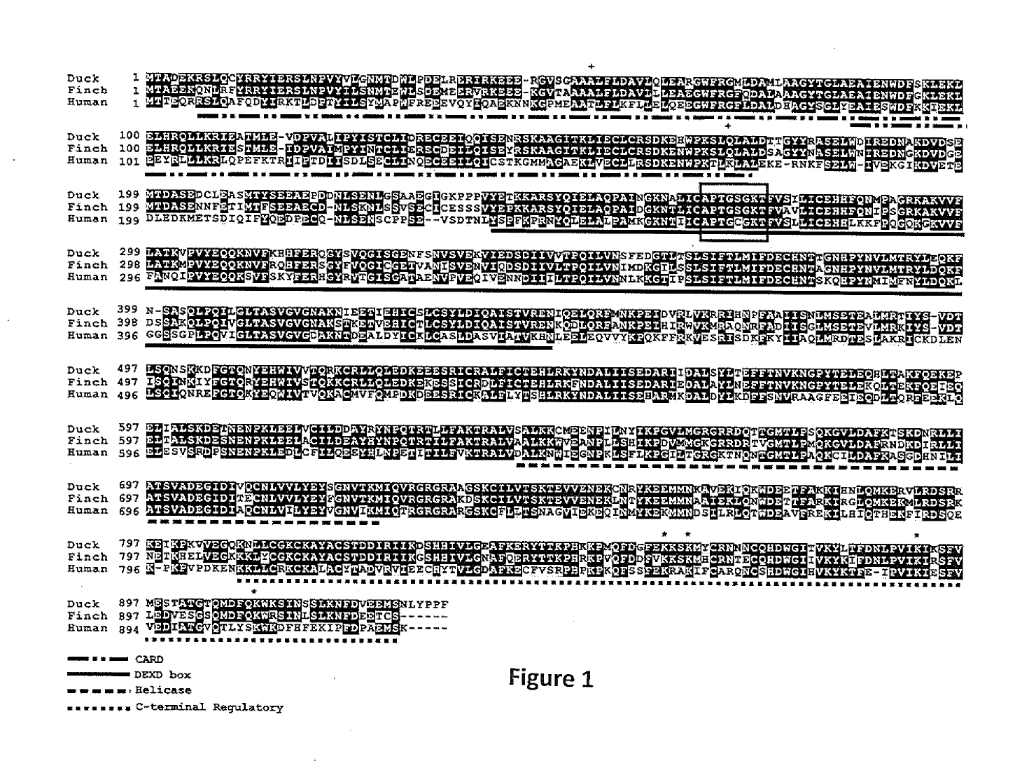

Given its role in antiviral defence in mammals, avian homologues of RIG-I were searched. A duck (Anas platyrynchos) RIG-I homologue with 53% amino acid identity to human RIG-I (FIG. 1) was identified. Duck RIG-I is 933 amino acids, and domain prediction reveals the expected tandem N-terminal CARD domains, a helicase domain, and a DeXD/H box helicase domain, consistent with the mammalian structure (Takahasi et al. 2008; Yoneyama et al. 2004), RIG-I is a ligand-dependent ATPase, and the Walker A ATP-binding motif is conserved. The hydrophobic core and the four lysine residues implicated in ligand-binding, K858/861/888/907 (Takahasi et al. 2008), are completely conserved within the C-terminal regulatory domain. However, residues T55 and K172, critical for attachment and polyubiquination by TRIM25 (Gack et al. 2007; Gack et al. 2008) are not conserved, suggesting this pathway does not function or involves different residues in ducks.

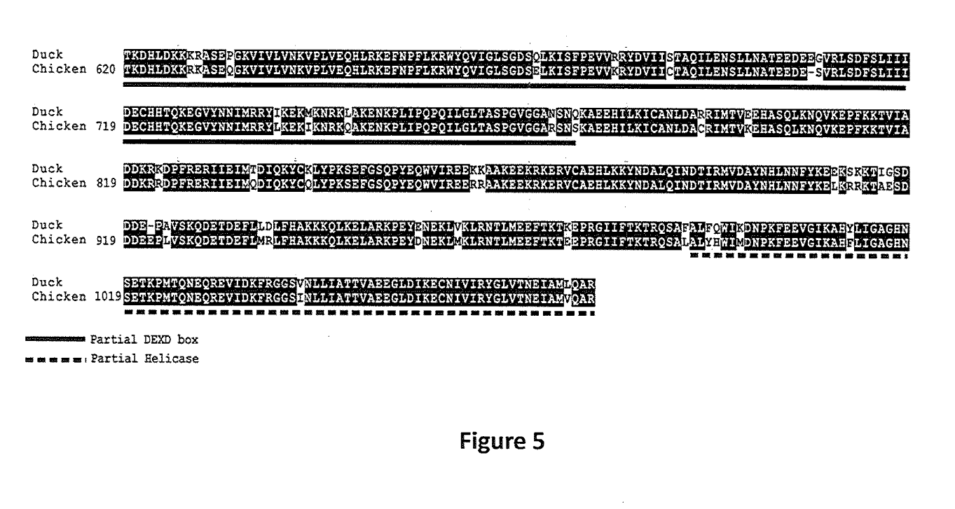

Remarkably, a chicken homologue of RIG-I was unable to be identified by a variety of approaches. Searches of the chicken (Gallus gallus) genome (International Chicken Genome Sequencing Consortium 2004) with the duck RIG-I sequence did not reveal a match. However, Melanoma Differentiation Associated gene-5 (MDA5) is present in chickens (Sarkar et al. 2008). RIG-I and MDA5 initiate signalling cascades that converge on the same pathway, but MDA5 is a detector of long dsRNA, polyinosinic-polycytidylic acid (poly (I:C)), and picornaviruses (Kato et al. 2006). To ensure that the isolated helicase was RIG-I, and distinct from MDA5, a large fragment of duck MDA5 was amplified. The fragment of MDA5 shared 91% amino acid identity to chicken MDA5 (FIG. 5), and only 33% identity to duck RIG-I.

The RIG-I synthetic region was identified on the chicken Z chromosome where acontinase I is encoded, a gene flanking the mammalian RIG-I homologue. However, a local BLAST search of the adjacent 4 Mb revealed no match to RIG-I. Confirming the synthetic region was conserved in other birds, a RIG-I homologue was identified in the recently released draft genome for zebrafinch (Taeniopygia guttata) on chromosome Z and flanked by acontinase I. Zebrafinch RIG-I has 78% amino acid identity to duck RIG-I (FIG. 1). A search of the finch expressed sequence tag (EST) database revealed two RIG-I transcripts among the 92,000 sequences. In contrast, no RIG-I sequences were present among the 600,000 ESTs from chicken. Notably, MDA5 transcripts were present. Thus, RIG-I was absent from the chicken genome sequence derived from the Red Jungle Fowl, which resembles the ancestral chicken, as well as the modern chicken lines represented in the EST sequences. Therefore, chickens might have lost RIG-I prior to their domestication.

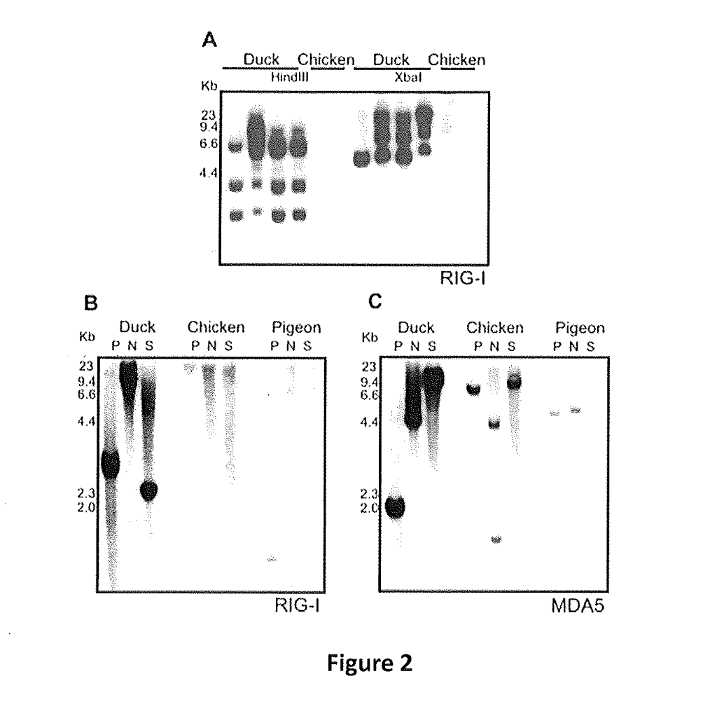

To confirm the absence of RIG-I in chicken, a Southern blot of genomic DNA from ducks and chickens was hybridized with a probe amplified from the helicase region of duck RIG-I (FIG. 2A). While a polymorphic pattern of hybridization was observed in ducks, no cross-hybridization to chicken DNA was detected. However, the duck probe cross-hybridized with RIG-I from the more phylogenetically distant pigeon (FIG. 2B), confirming it can recognize RIG-I from other avian species. It is noteworthy that pigeons are remarkably resistant to influenza viruses, including H5N1 (Perkins and Swayne 2002). In comparison, strong cross-hybridization of a duck MDA5 probe with DNA from pigeon and chicken (FIG. 2C) suggests it has diverged considerably less than RIG-I and is presumably under less selective pressure in avian species.

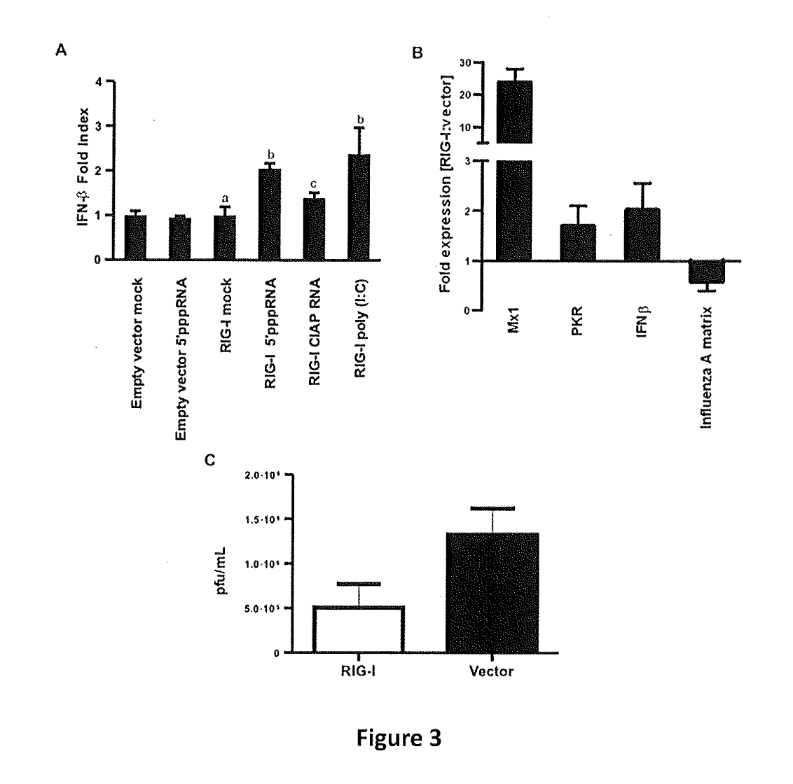

The apparent absence of RIG-I in chickens led to investigate whether chicken cells can respond to a RIG-I ligand. RIG-I signalling is activated by 5′ppp RNA containing short RNA with double-strand conformation, such as that derived from viral RNA with panhandle structures. (Schlee et al, 2009; Schmidt et al. 2009), and from in vitro transcribed products (Hornung et al. 2006; Pichlmair et al. 2006). Challenge of the DF-1 cell line (chicken embryonic fibroblasts) with an in vitro transcribed 21mer 5′ppp RNA failed to activate a chicken IFN-β promoter-luciferase reporter (FIG. 3A). The MDA5 ligand; poly (I:C), known to drive the chicken IFN-β promoter in this cell line (Childs et al. 2007), showed a 2.4 fold increase in IFN-β promoter activity, compared to unstimulated cells. That DF-1 cells responded to poly (I:C) with an interferon response, demonstrated that they possess the downstream components of the shared RIG-I/MDA5 pathway. To determine whether duck RIG-I could confer recognition of a RIG-I ligand, DF-1 cells were transfected with duck RIG-I prior to stimulation with 5′ppp RNA. DF-1 cells expressing duck RIG-I responded to 5′ppp RNA with 2 fold induction of the IFN-β promoter, as compared to mock-transfected cells. Phosphatase removal of the 5′ triphosphate abrogated the response, as in mammalian cells (Hornung et al. 2006).

To determine if duck RIG-I could detect influenza virus and induce an antiviral response in the chicken DF-1 cells, the cells with duck RIG-I or vector only were transfected, followed by infection with H5N2 A/mallard/BC/2005, a low pathogenic avian influenza (LPAI) isolated from wild ducks that induces no pathology to its natural host. After 15 hours, there was increased expression of IFN-β as well as the antiviral interferon-stimulated genes Mx1 and PKR, known to be RIG-I responsive in mouse fibroblasts (Loo et al. 2008) (FIG. 3B). Influenza virus titre and influenza A matrix gene expression were significantly reduced in RIG-I transfected DF-1 cells (FIG. 3B,C), indicating that duck RIG-I can reduce influenza replication in chicken cells.

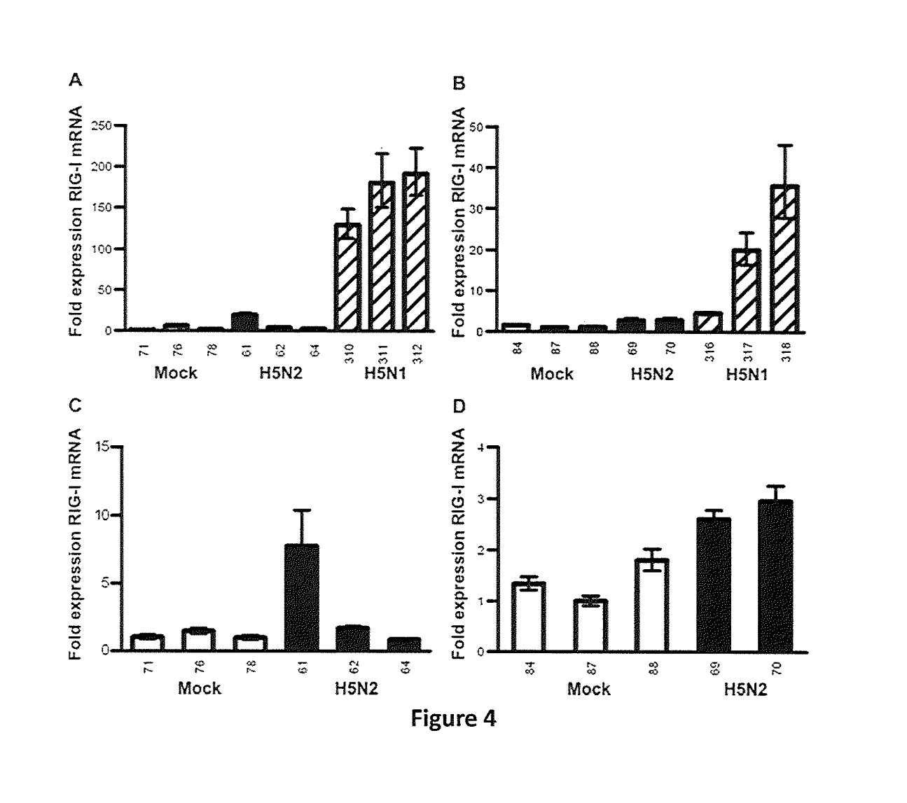

To determine whether RIG-I is involved in an antiviral response to influenza in ducks, the expression of RIG-I was investigated in 6-week-old White Pekin duck tissues following influenza infections with H5N2 or H5N1 A/Vietnam/1203/04, a HPAI human isolate known to be lethal to ducks, chickens and humans (Hulse-Post et al. 2007). Infection with H5N1 induced significant upregulation of RIG-I gene expression in the infected lung tissue (FIG. 4A). RIG-I was upregulated over 200-fold by d1 PI, however by d3 PI, RIG-I was only modestly upregulated, suggesting the induction is early and transient (FIG. 4B). In comparison, infection with H5N2 induced only slight upregulation of RIG-I in lung tissue. Because LPAI strains replicate within the intestinal epithelium of ducks (Webster et al. 1978), RIG-I expression was also assessed in H₅N₂ infected duck intestine (FIG. 4C, D). RIG-I expression was not significantly upregulated by H5N2 infection in duck intestine on d1 or d3 PI. It is not clear why H₅N₁ results in tremendous upregulation of RIG-I, while H5N2 does not. Influenza strains vary in their ability to actively inhibit interferon induction in birds and mammals, a function which is dependent on the viral protein, NS1 (Egorov et al. 1998; Garcia-Sastre et al. 1998; Li et al. 2006), which directly targets the RIG-I pathway preventing activation of IFN-β (Mibayashi et al. 2007). Without wishing to be bound by theory, one possibility is that the NS1 protein of H5N2 interferes with viral activation of the interferon pathway, while the NS1 of H5N1 virus does not. Although the H5N1 virus is potentially lethal in ducks (Hulse-Post et al. 2007), none of the ducks showed severe symptoms or died, implying that the robust and transient upregulation of RIG-I was part of a successful innate response.

### Discussion

The results herein show RIG-I is involved in the innate immune response of ducks to influenza virus, and provide natural evidence of the significance of RIG-I for antiviral protection. Ducks have a functional RIG-I that induces IFN-β promoter activity and antiviral responses upon ligand detection, and is upregulated during HPAI infection. RIG-I appears to be absent in chickens and a chicken embryonic fibroblast cell line cannot respond to 5′ppp RNA, a function that can be conferred by transfection with duck RIG-I. Expression of duck RIG-I reduces influenza replication in chicken cells. Although chicken embryonic fibroblasts infected with influenza do produce interferons and inhibit an IFN-sensitive vesicular stomatitis virus (VSV)-GFP (Li et al. 2006), this response is 80% IFN-α (Schwarz et al. 2004). IFN-β expression upon influenza infection is largely dependent on RIG-I. RIG-I knockout mouse embryonic fibroblasts fail to induce IFN-β and a subset of genes involved in innate immunity in response to influenza infection (Loo et al. 2008). siRNA knockdown of RIG-I (Opitz et al. 2007) or introduction of a dominant negative RIG-I (Siren et al. 2006) significantly reduced the influenza-induced IFN-β production in human cell lines. Additionally, IFN-β knockout mice show reduced survival and enhanced influenza viral titres in the lung (Koerner et al. 2007). Thus, IFN-β is protective in an influenza infection and cannot be compensated by IFN-α. Newcastle Disease Virus (NDV), also detected by RIG-I, causes substantially more pathology in chickens than ducks. NDV infection does not induce IFN-β promoter activity in chicken embryonic fibroblasts (Sick et al. 1998), also consistent with lack of RIG-I. All interferon produced by chicken embryo cells in response to NDV is IFN-α (Schwarz et al. 2004). Chickens without RIG-I would lack the first line of defence at the mucosal epithelial cell layer, and have impaired recruitment of leukocytes to infected tissues. Antiviral genes downstream of RIG-I may not be expressed (Loo et al. 2008). Since RIG-I may also inhibit influenza replication directly, independent of IFN (Yoneyama et al. 2004), loss of this undefined pathway might also contribute to unchecked viral replication in chickens. The presence of a 5′ppp RNA sensor in duck cells eliciting IFN-β production and an antiviral response undoubtedly contributes to the ability of ducks to survive lethal influenza infection.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Childs, K. et al., Virology 359, 190-200 (2007).
Egorov, A. at al., J. Virol. 72, 6437-6441 (1998).
Gack, M. et al., Nature 446, 916-920 (2007).
Gack, M, at al., Proc. Natl. Acad. Sci. U.S.A. 105, 16743-16748 (2008),
Garcia-Sastre, A. et al., Virology 252, 324-330 (1998).
Himly, M., D. N. Foster, I. Bottoli, J. S. lacovoni, P. K. Vogt, Virology 248, 295-304 (1998).
Hornung, V. at al., Science 314, 994-997 (2006).
Hulse-Post, D. et al., Proc. Natl. Acad. Sci. U.S.A. 102, 10682-10687 (2005).
Hulse-Post, D. et al., J. Virol. 81, 8515-8524 (2007).
International Nature 432, 695-716 (2004).
Kato, H. et al., Nature 441, 101-105 (2006),
Kim, J. H., N. J. Negovetich, H. L. Forrest, R. G. Webster, Influenza Other Respi. Viruses. 3, 121-128 (2009).
Kobasa, D. et al., Nature 445, 319-323 (2007).
Koerner, I., G. Kochs, U. Kalinke, S. Weiss, P. Staeheli, J. Virol. 81, 2025-2030 (2007).
Li, Z. et al., J. Virol. 80, 11115-11123 (2006).
Loo, Y. M. et al., J. Virol. 82, 335-345 (2008).
Mibayashi, M. et al., J. Virol. 81, 514-524 (2007).
Opitz, B. et al., Cell Microbiol. 9, 930-938 (2007).
Perkins, L., D. E. Swayne, Avian Dis. 46, 53-63 (2002).
Peters, M. A., G. F. Browning, E. A. Washington, B, S. Crabb, P. Kaiser, Immunology 110, 358-367 (2003).
Pichlmair, A. et al., Science 314, 997-1001 (2006).
Salomon, R. et al., J. Exp. Med. 203, 689-697 (2006).
Sarkar, D., R. DeSalle, P. B. Fisher, Proc. Natl. Acad. Sci. U.S.A. 105, 17040-17045 (2008).
Schlee, M. et al., Immunity 31, 25-34 (2009).
Schmidt, A. at al., Proc. Natl. Acad. Sci. U.S.A. 106, 12067-12072 (2009).
Schwarz, H., O. Harlin, A. Ohnemus, B. Kaspers, P. Staeheli, J. Interferon Cytokine Res. 24, 179-184 (2004).
Sick, C. et al., J. Biol. Chem., 273, 9749-9754 (1998).
Sirén, J. et al., Microb. Infect. 8, 2013-2020 (2006).
Sturm-Ramirez, K. et al., J. Virol. 79, 11269-11279 (2005).
Takahasi, K. at al., Mol. Cell. 29, 428-440 (2008).
Yoneyama, M. et al., Nature Immunol. 5, 730-737 (2004).
Webster; R. G., M. Yakhno, V. S. Hinshaw, W. J. Bean, K. G. Murti, Virology 84, 268-278 (1978).
Webster, R. G., W. J. Bean, O. T. Gorman, T. M. Chambers, Y. Kawaoka, Microbiol. Mol. Biol. Rev. 56, 152-179 (1992).

1. A chicken cell transformed with a recombinant nucleic acid molecule encoding a RIG-I protein, a functional variant or a fragment thereof.

2. The chicken cell of claim 1, wherein the RIG-I protein is at least one of duck RIG-I, pigeon RIG-I or zebra finch RIG-I.

3. The chicken cell of claim 2, wherein when the RIG-I protein is duck RIG-I, the duck RIG-I comprises the amino acid sequence of SEQ ID NO:1 or a protein substantially identical in biological activity thereto, and further wherein when the RIG-1 protein is zebra finch RIG-I, the zebra finch RIG-I comprises the amino acid sequence of SEQ ID NO:2 or a protein substantially identical in biological activity thereto.

4. The chicken cell of claim 3, wherein the duck RIG-I is encoded by the nucleic acid sequence of SEQ ID NO:30 and the zebra finch RIG-I is encoded by the nucleic acid sequence of SEQ ID NO:31.

5. The chicken cell of claim 1, wherein the recombinant nucleic acid molecule is a plasmid or viral vector.

6. The chicken cell of claim 5, wherein the viral vector is a retroviral vector, an adenoviral vector or an adeno-associated viral vector.

7. A transgenic chicken comprising a chicken cell according to claim 1.

8. The transgenic chicken of claim 7, wherein the recombinant nucleic acid molecule is integrated into the genome of said chicken.

9. The transgenic chicken of claim 8, wherein the recombinant nucleic acid molecule is expressed in its germ cells and somatic cells.

10. A method of reducing RNA virus replication in a chicken cell comprising expressing a recombinant nucleic acid molecule encoding a RIG-I protein or functional variant or fragment thereof in the chicken cell.

11. The method according to claim 10, wherein the RIG-I protein is at least one of duck RIG-I, pigeon RIG-I or zebra finch RIG-I.

12. The method according to claim 11, wherein when the RIG-I protein is duck RIG-I, the duck RIG-I comprises the amino acid sequence of SEQ ID NO:1 or a protein substantially identical in biological activity thereto, and further wherein when the RIG-1 protein is zebra finch RIG-I, the zebra finch RIG-I comprises the amino acid sequence of SEQ ID NO:2 or a protein substantially identical in biological activity thereto.

13. The method according to claim 12, wherein the duck RIG-I is encoded by the nucleic acid sequence of SEQ ID NO:30 and the zebra finch RIG-I is encoded by the nucleic acid sequence of SEQ ID NO:31.

14. The method according to claim 10, wherein the recombinant nucleic acid molecule is a plasmid or viral vector.

15. The method of claim 10, wherein the RNA virus is a negative single stranded RNA virus.

16. The method of claim 15, wherein the negative single stranded virus is influenza or Newcastle Disease virus.

17. A method of producing a transgenic chicken with increased resistance to RNA virus infection, comprising:
a) inserting into a chicken embryo a recombinant nucleic acid molecule comprising a foreign RIG-I gene; and
b) allowing the embryo to hatch.

18. The method of claim 17, wherein (a) comprises (i) inserting into a chicken germline or embryonic stem cell a recombinant nucleic acid molecule comprising a foreign RIG-I, (ii) producing a zygote from said germline or embryonic stem cell and (iii) allowing the zygote to form an embryo.

19. The method of claim 18, wherein the chicken germline cell is a sperm cell or an egg cell.

20. The method of claim 18, wherein the chicken germline cell is a primordial germ cell.
